(19) United States
(12) Patent Application Publication
(10) Pub. No.: US 2008/0188825 A1
(43) Pub. Date: Aug. 7, 2008

(54) Catheters and medical balloons

(76) Inventors: Liliana Atanasoska, Edina, MN (US); Jan Weber, Maastricht (NL); Scott R. Schewe, Eden Prairie, MN (US); Robert W. Warner, Woodbury, MN (US); John Chen, Plymouth, MN (US)

Correspondence Address: FISH & RICHARDSON PC, P.O. BOX 1022, MINNEAPOLIS, MN, 55440-1022, US

(21) Appl. No.: 11/701,148

(22) Filed: Feb. 1, 2007

(51) Int. Cl. A61M 25/10

(52) U.S. Cl. 604/509

(57) ABSTRACT

Medical balloons and/or catheters are disclosed that include a wall that includes a composite material. The composite material includes a polymeric material and particles that include an allotrope of carbon. In some instances, at least some of the particles are covalently bonded to the polymeric material. Methods for making such medical balloons and/or catheters are also disclosed.

### TECHNICAL FIELD

This disclosure relates to catheters and medical balloons, and to methods of making the same.

### BACKGROUND

The body includes various passageways such as arteries, other blood vessels, and other body lumens. These passageways sometimes become occluded, e.g., by a tumor or restricted by plaque. To widen an occluded body vessel, balloon catheters can be used, e.g., in angioplasty.

A balloon catheter can include an inflatable and deflatable balloon carried by a long and narrow catheter body. The balloon is initially folded about the catheter body to reduce the radial profile of the balloon catheter for easy insertion into the body.

During use, the folded balloon can be delivered to a target location in the vessel, e.g., a portion occluded by plaque, by threading the balloon catheter over a guide wire emplaced in the vessel. The balloon is then inflated, e.g., by introducing fluid into the interior of the balloon. Inflating the balloon can radially expand the vessel so that the vessel can permit an increased rate of blood flow. After use, the balloon is deflated and withdrawn from the body.

In another technique, the balloon catheter can also be used to position a medical device, such as a stent or a stent-graft, to open and/or to reinforce a blocked passageway. For example, the stent can be delivered inside the body by a balloon catheter that supports the stent in a compacted or reduced-size form as the stent is transported to the target site. Upon reaching the site, the balloon can be inflated to deform and to fix the expanded stent at a predetermined position in contact with the lumen wall. The balloon can then be deflated and the catheter withdrawn. Stent delivery is further discussed in Heath, U.S. Pat. No. 6,290,721.

One common balloon catheter design includes a coaxial arrangement of an inner tube surrounded by an outer tube. The inner tube typically includes a lumen that can be used for delivering the device over a guide wire. Inflation fluid passes between the inner and outer tubes. An example of this design is described in Arney et al., U.S. Pat. No. 5,047,045.

In another common design, the catheter includes a body defining a guide wire lumen and an inflation lumen arranged side-by-side. Examples of this arrangement are described in Wang et al., U.S. Pat. No. 5,195,969.

### SUMMARY

This disclosure relates to catheters and medical balloons, and to methods of making the same.

In one aspect, the disclosure features medical balloons and/or catheters that include a wall that includes a composite material. The composite material includes a first polymeric material and first particles that include an allotrope of carbon. At least some of the first particles are bonded, e.g., covalently bonded, to the first polymeric material. Bonding, e.g., covalently bonding and/or hydrogen bonding, the first particles to the first polymeric material can improve dispersion and can reduce particle aggregation and/or phase separation of the first particles in the polymeric material of the composite.

Embodiments may have one or more of the following features. The first polymeric material includes segments including polyethers, polyurethanes, polyether-polyurethane copolymers, polyamides polyether-polyamide copolymers, polyureas, polyether-polyurea copolymers, polyamines, polyesters, polysiloxanes or mixtures thereof. The first polymeric material includes a thermoplastic material. The first polymeric material includes a crosslinked material. The composite material further includes a second polymeric material different than the first polymeric material. The particles are fibrous in form. The particles are tubular in form. The particles are in the form of coils. The allotrope of carbon includes graphite, C60, C70, a single wall carbon tube, multi-wall carbon tube, amorphous carbon, a carbon coil, a carbon helix, carbon rope, carbon fiber or mixtures thereof. Each first particle has a length-to-diameter ratio of greater than 5. Each first particle has a length-to-diameter ratio of greater than 25. Each first particle has a maximum dimension not exceeding 2,000 nm. Each first particle has a maximum dimension not exceeding 1,000 nm. The first particles are discrete and spaced apart throughout the composite. The composite further includes second particles different than the first particles. The second particles may or may not be covalently bonded to the first polymeric material. The second particles are metals, metal oxides, metalloid oxides, clays, ceramics or mixtures thereof. At least about 2.5 percent of the total number of the first particles are covalently bonded to the first polymeric material. At least about 25 percent of the total number of the first particles are covalently bonded to the first polymeric material. The composite includes from about 5 percent by weight to about 60 percent by weight of the first particles including the allotrope of carbon. The composite includes from about 15 percent by weight to about 50 percent by weight of the first particles including the allotrope of carbon. The first particles are covalently bonded to the first polymeric material by a covalent bond connecting a carbon atom of the allotrope of carbon and the first polymeric material. The first particles are covalently bonded to the first polymeric material by a reaction between a nucleophilic moiety covalently attached to the allotrope of carbon and a complementary electrophilic moiety covalently attached to the first polymeric material or a pre-first polymeric material. The first particles are covalently bonded to the first polymeric material by a reaction between an electrophilic moiety covalently attached to the allotrope of carbon and a complementary nucleophilic moiety covalently attached to the first polymeric material or a pre-first polymeric material. The nucleophilic moiety includes a nucleophile selected from an amino group, a hydroxyl group, a thiol group, conjugate bases thereof or mixtures thereof. The electrophilic moiety includes an electrophile selected from a carboxylic acid group, an ester group, a thioester group, an amide group, a urethane group, a urea group or mixtures thereof. Each first particle has between about 2 and about 1,000 nucleophilic and/or electrophilic moieties. At least some of the first particles including the allotrope of carbon further include a substrate bonded to the allotrope of carbon. The support includes a clay including an allotrope of carbon-forming catalyst thereon and/or therein. The clay includes kaolinite, montmorillonite-smectite, illite, chlorite or mixtures thereof. The clay includes montmorillonite. The wall is or includes multiple layers. The composite material is in each layer of the multiple layers, and each layer is integral with its neighbor. The composite material is in a single layer, the single layer being integral with a second layer formed of a material different than the composite material. The wall includes a therapeutic agent therein and/or thereon.

In another aspect, the disclosure features methods of making medical balloons and/or catheters that include forming a wall that includes a composite material that includes a first polymeric material and first particles that include an allotrope of carbon.

Embodiments may have one or more of the following features. The wall is formed by providing a substrate; and depositing the composite material onto the substrate. The method further includes removing the substrate. The substrate includes ice. The composite material is deposited by spraying a solution of the composite material onto the substrate. The method further includes repeating the spraying.

In another aspect, the disclosure features medical balloons and/or catheters that include a wall that includes a composite material that includes a polymeric material and particles. The particles include a substrate that includes a material that includes a clay, and an allotrope of carbon extending from the substrate.

Embodiments may have one or more of the following features. The clay has an allotrope of carbon-forming catalyst thereon and/or therein. The clay includes kaolinite, montmorillonite-smectite, illite, chlorite or mixtures thereof. The clay includes montmorillonite. The allotrope of carbon-forming catalyst includes a group 8 or group 9 element. The allotrope of carbon-forming catalyst is or includes iron.

Embodiments and/or aspects may include one or more of the following advantages. Balloons and catheters can be provided that are formed of a composite material in which particles of the composite material are evenly dispersed throughout the polymeric material of the composite and are not excessively aggregated. This can provide balloons and/or catheters with properties that are uniform and reproducible. Balloons can be provided in which properties, such as puncture resistance, scratch resistance, burst strength, tensile strength, porosity, drug release, and electrical and thermal conductivity, are enhanced for a given application. Balloons and/or catheters can be thermally and/or electrically conductive. The composite materials can have a high tensile strength, e.g., greater 100 MPa, e.g., greater than 150, 250, or even greater than 300 MPa, enabling thin and ultra-thin walled balloons and/or catheters. The composites can be thermoplastic or thermoset. The polymeric material of the composite can be linear, branched, highly branched or dendritic in nature, allowing the composite to have properties that are tailored for a given application. The catheters and/or balloons can have enhanced biocompatibility.

All publications, patent applications, patents, and other references mentioned herein are incorporated by reference herein in their entirety for all that they contain.

The details of one or more embodiments of the disclosure are set forth in the accompanying drawings and the description below. Other features and advantages of the disclosure will be apparent from the description and drawings and from the claims.

### DESCRIPTION OF DRAWINGS

FIGS. 1A-1C are partial longitudinal cross-sectional views, illustrating delivery of a stent in a collapsed state (FIG. 1A), expansion of the stent (FIG. 1B), and deployment of the stent in a body lumen (FIG. 1C).

FIG. 2 is a highly enlarged, schematic representation of a medical balloon and/or catheter wall that includes a composite material that includes a polymeric material and particles that include an allotrope of carbon.

FIG. 3A is a schematic representation of a functionalized particle, while FIG. 3B is a schematic representation of a particle functionalized with carboxylic acid groups.

FIGS. 4A-4G are scanning electron micrographs of various carbon coils.

FIG. 4H is a schematic representation of a particle that includes a substrate and a carbon coil extending from the substrate.

FIG. 4I is a schematic representation of the particle of FIG. 4H functionalized with carboxylic acid groups on the coil and substrate.

FIG. 5 is a schematic representation of several synthetic strategies for producing carboxylic acid group-functionalized carbon coils.

FIG. 6A is a schematic representation of a synthetic strategy for producing acid chloride-functionalized carbon coils from carboxylic acid group-functionalized carbon coils.

FIG. 6B is a schematic representation of several synthetic methods for producing derivatives of carboxylic acid group-functionalized carbon coils.

FIG. 7 is a schematic representation of carboxylic acid group-functionalized carbon coils reacting with toluene-2,4-di-isocyanate.

FIG. 8 is a schematic representation for the preparation of a pre-polymer from the reaction product of FIG. 7 and a polyol.

FIGS. 9A-9D show representative structures of some polyols.

FIG. 10 is a schematic representation of the preparation of high molecular weight isocyanate-terminated polymer.

FIG. 11 is a schematic representation of the preparation of high molecular weight, end-capped polymer from the polymer of FIG. 10 and isopropanol.

FIG. 11A is a schematic representation of hydrogen bonding sites in the polymer shown in FIG. 11.

FIG. 12 is a schematic representation of the preparation of a trialkoxy-terminated silane polymer.

FIG. 13 is a schematic representation of the carboxylic acid group-functionalized carbon coil/toluene-2,4-di-isocyanate reaction product of FIG. 8 reacting with various polyamine materials (FIGS. 13A-13F).

FIG. 14 is a schematic representation of the preparation of a trialkoxy-terminated silane polymer and its crosslinking.

FIG. 15 is a schematic representation of the preparation of a isocyanate-terminated pre-polymer by reaction of a polyol with hexamethylene di-isocyanate.

FIG. 16 is a schematic representation of a acid chloride-functionalized carbon coil reacting with a polyol to generate a pre-polymer having ester groups.

FIG. 17 is a schematic representation of a method for making the balloon of FIG. 1A.

### DETAILED DESCRIPTION

Medical balloons and/or catheters are disclosed that include a wall that includes a composite material. The composite material includes a polymeric material and particles that include an allotrope of carbon. In some instances, at least some of the particles are bonded, e.g., covalently bonded or hydrogen bonded, to the polymeric material. Methods for making such medical balloons and catheters are also disclosed.

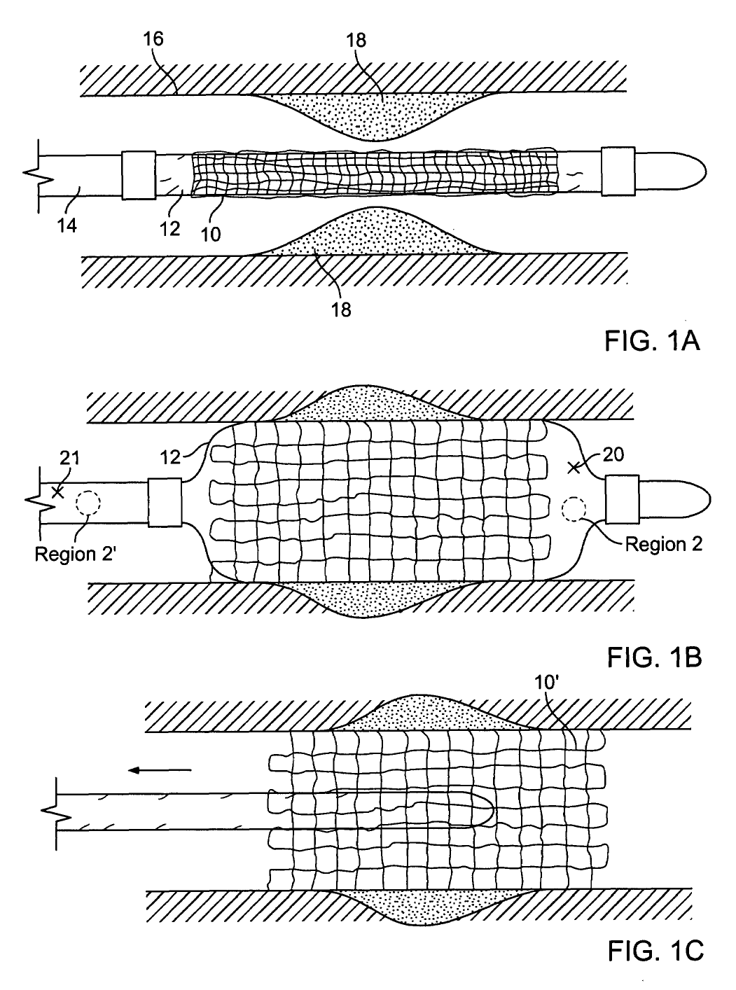

Referring to FIGS. 1A-1C, an unexpanded stent 10 is placed over a balloon 12 carried near a distal end of a catheter 14, and is directed through a lumen 16, e.g., a blood vessel such as the coronary artery, until the portion carrying the balloon and stent reaches the region of an occlusion 18 (FIG. 1A). The stent is then radially expanded by inflating the balloon 12, and is pressed against the vessel wall with the result that occlusion 18 is compressed and the vessel wall surrounding it undergoes a radial expansion (FIG. 1B). The pressure is then released from the balloon and the catheter is withdrawn from the vessel, leaving behind expanded stent 10′ in the lumen (FIG. 1C).

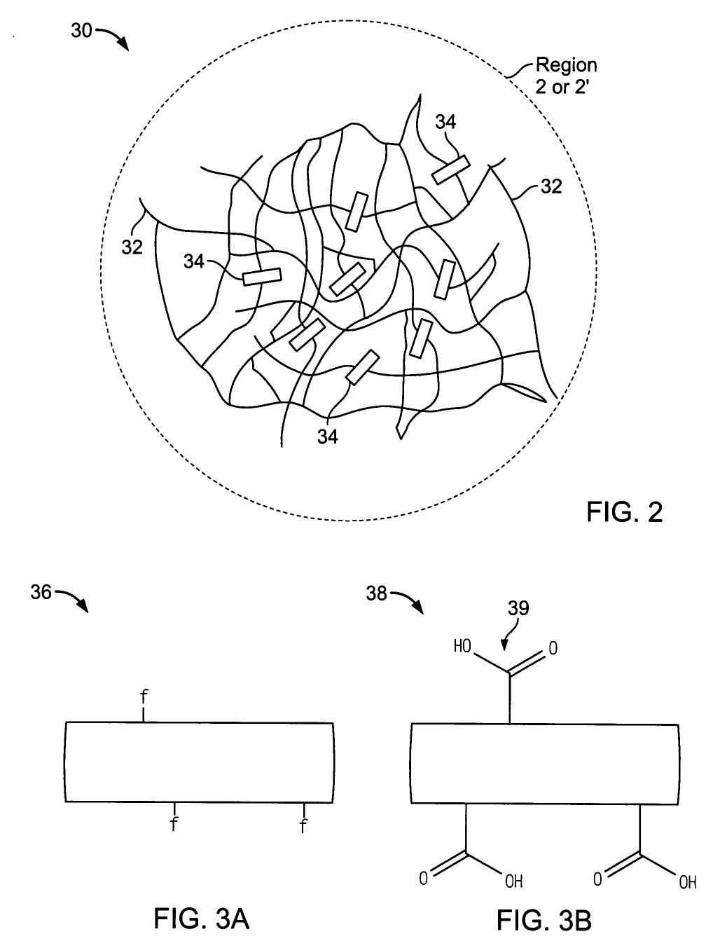

Referring also now to FIG. 2, the catheter 14 and/or balloon 12 includes a wall 21 or 20, respectively, formed of a composite material 30 that includes a first polymeric material 32 and first particles 34. The first particles 34 include an allotrope of carbon, e.g., carbon coils or carbon helices, and are uniformly dispersed within the first polymeric material 32. At least some of the first particles 34 are covalently bonded to the first polymeric material 32. Referring also now to FIGS. 3A and 3B, the first particles 34 can be covalently bonded to the first polymeric material 34, or a material that will become part of the first polymeric material by using a particle 36 having a functional moiety (ƒ), e.g., a nucleophilic or an electrophilic moiety. For example, and as will be discussed in further detail below, a particle 38 having a plurality of carboxylic acid groups 39 can be grafted onto a polymeric matrix by reaction with a complementary moiety, e.g., a moiety that includes one or more isocyanate groups, that is part of the polymeric matrix or a pre-polymeric matrix material.

In embodiments, the first polymeric material includes polymer segments which are polyethers, polyurethanes, polyether-polyurethane copolymers, polyamides, polyether-polyamide copolymers (e.g., PEBAX® brand polyether-block-polyamides), polyureas, polyether-polyurea copolymers, polyamines, polyesters (e.g., PET), polysiloxanes, or mixtures of any of these.

In embodiments, the first polymeric material is a thermoplastic material, allowing the composite material to be processed using thermoplastic processing equipment, e.g., extrusion equipment, injection molding equipment, blow molding equipment or roto-molding equipment. When the composite material is a thermoplastic material, it can also be dissolved in a solvent and cast or coated onto a substrate, e.g., a substrate made of another polymeric material.

In other embodiments, the first polymeric material is a crosslinked material.

In still other instances, the first polymeric material is initially a thermoplastic, and then after a wall is formed, the first polymeric is crosslinked, e.g., by treatment with ionizing radiation such as gamma radiation.

If desired, the composite material can further include a second, third, fourth, or even a fifth polymeric material different than the first polymeric material.

In embodiments, the particles are fibrous in form or tubular in form. In other embodiments, the particles are in the form of coils.

Each first particle can, e.g., have a length-to-diameter ratio of greater than 5, e.g., greater than 10, greater than 25, greater than 50, greater than 100 or even greater than 250.

Each first particle can have a maximum dimension not exceeding 6,000 nm, e.g., not exceeding 5,000 nm, not exceeding 2,500 nm, not exceeding 2,000 nm, not exceeding 1,500 nm, not exceeding 1,000 nm, not exceeding 750 nm or not exceeding 500 nm. In embodiments, the maximum dimension of each first particle is less than 250 nm, e.g., less than 200 nm, less than 150 nm or even less than 100 nm.

In embodiments, the allotrope of carbon is inherently thermally and/or electrically conductive. In embodiments, the allotrope of carbon is doped, e.g., with one or more metals, so it becomes electrically and/or thermally conductive. In such instances, the composites and balloons and/or catheters that are formed from the composite can be made electrically and/or thermally conductive.

The allotrope of carbon can be, e.g., graphite, C60, C70, a single wall carbon tube, a multi-wall carbon tube, amorphous carbon, a carbon coil, a carbon helix (e.g., a chiral right-handed or left-handed helix), carbon rope, carbon fiber or mixture of these. If desired, the carbon nanotubes can encapsulate atoms other than carbon, such as metal. In embodiments, the allotrope of carbon contains greater than 90 percent by weight carbon, e.g., greater than 91, 93, 95, 97, or even greater than 99 percent carbon by weight. In embodiments, the allotrope of carbon is formed substantially of carbon, having only bound hydrogen at boundaries.

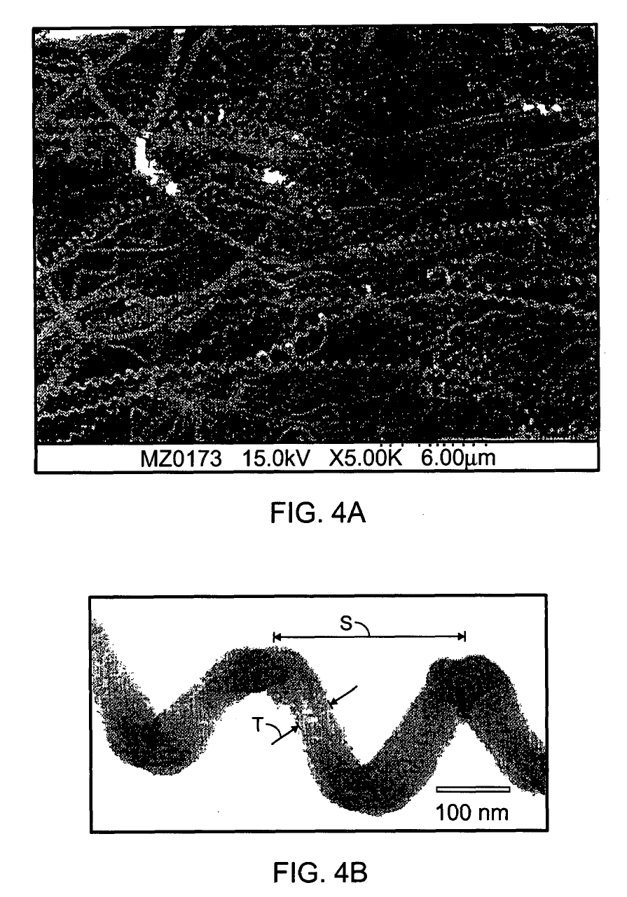

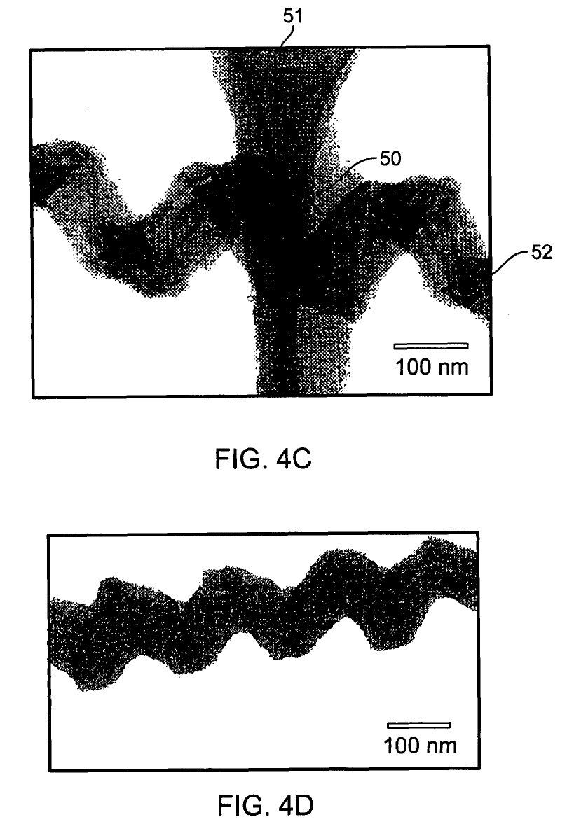

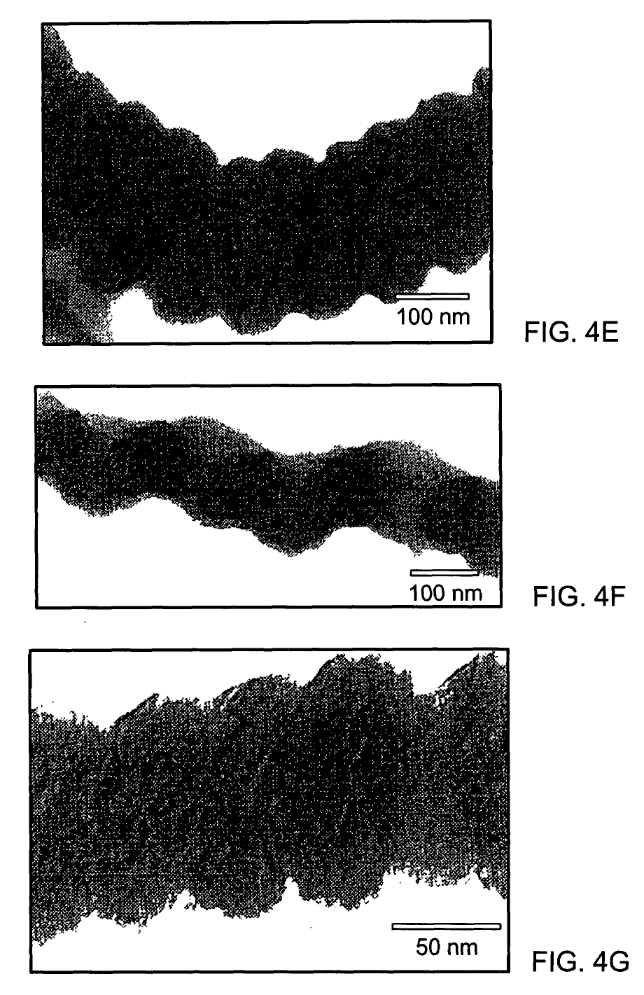

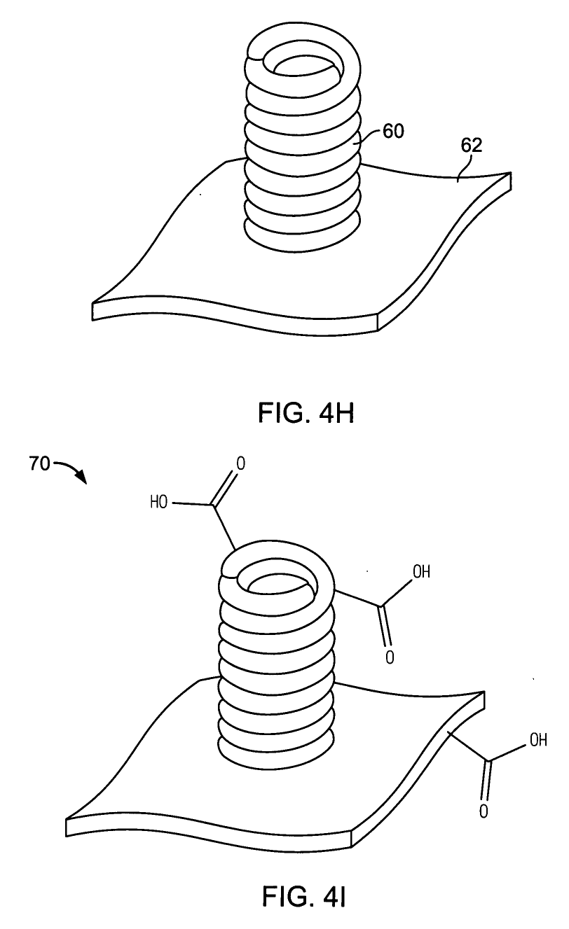

FIGS. 4A-4G are scanning electron micrographs of various carbon coils. In particular, FIGS. 4A-4D show carbon coils in which the turns are relatively spaced apart so that there is open space between turns, while FIGS. 4E-4G show carbon coils having a relatively dense structure in which the turns of the coils are touching adjacent turns. FIG. 4C shows that the carbon coils can have branch points 50 in which other coils 51 emanate from a central coil 52. In embodiments, spacing (S) between turns in the carbon coils can range from about 10 nm to about 250 nm, e.g., between about 20 nm and about 100 nm or between about 25 nm and about 75 nm. In embodiments, the thickness (T) of the rod forming the coil is between about 20 nm and about 100 nm, between about 25 nm and about 80 nm or between about 30 nm and about 75 nm. Methods of making unfunctionalized carbon coils are discussed in Nakayama et al., U.S. Pat. Nos. 7,014,830, 6,558,645 and 6,583,085; Deck et al., Carbon 44 (2006), 267-275; and in Yang et al., Carbon 43 (2005), 916-922.

In some embodiments, carbon nanotubes are utilized. Various carbon nanotubes, and some of their properties are described by Moulton et al., Carbon, 43, 1879-1884 (2005); Jiang et al., Electrochemistry Communications, 7, 597-601 (2005); and Shim et al., Langmuir, 21(21), 9381-9385 (2005).

Referring to FIG. 4H, in embodiments, at least some of the first particles include an allotrope of carbon in the form of coil 60 extending from a substrate 62, which includes a clay material. In such embodiments, the clay can be a kaolinite clay, montmorillonite-smectite, an illite clay, a chlorite clay or mixtures of these clays. Substrate 62 can, e.g., include a clay that includes an allotrope of carbon-forming catalyst thereon and/or therein. Referring also now to FIG. 4I, such particles can be covalently bonded to the first polymeric material or a material that will become part of the first polymeric material by using a particle 70 having a functional moiety (ƒ), e.g., a carboxylic acid group covalently bonded to the allotrope of carbon and/or the substrate. Such carboxylic acid groups can be grafted onto a polymeric matrix by reaction of a complementary moiety, e.g., a moiety that includes one or more isocyanate groups, that is part of the polymeric matrix of a pre-polymeric matrix material. Methods of making various particles that include a clay substrate having an allotrope of carbon extending therefrom are discussed in Lu et al, Composites Science and Technology 66 (2006), 450-458 and Carbon 44 (2006), 381-392.

In embodiments, at least about 2.5 of the total number of the first particles are covalently bonded to the first polymeric material, e.g., at least about 15 percent, at least about 25 percent, at least about 50 percent, at least about 75 percent, at least about 90 percent of the first particles are covalently bonded to the first polymeric material.

The composite can include, e.g., from about 15 percent by weight to about 75 percent by weight of the first particles, e.g., between about 15 percent by weight to about 50 percent by weight or between about 25 percent by weight to about 45 percent by weight.

In embodiments, the first particles are covalently bonded to the first polymeric material by a covalent bond connecting a carbon atom of the allotrope of carbon and the first polymeric material.

In embodiments, the first particles are covalently bonded to the first polymeric material by a reaction between a nucleophilic moiety covalently attached to the allotrope of carbon and a complementary electrophilic moiety covalently attached to the first polymeric material or a material that will become part of the first polymeric material (e.g., a pre-polymer). In other embodiments, the first particles are covalently bonded to the first polymeric material by a reaction between an electrophilic moiety covalently attached to the allotrope of carbon and a complementary nucleophilic moiety covalently attached to the first polymeric material or material that will become part of the first polymeric material (e.g., a pre-polymer).

For example, the nucleophilic moiety can include a nucleophile, such as an amino group, a hydroxyl group, a thiol group, a carboxylic acid group, a conjugate base of any of these or mixtures of any of these. For example, the electrophilic moiety can include an electrophile, such as a carboxylic acid group, an isocyanate group, an ester group, a thioester group, an amide group, a urethane group, a urea group or mixtures of any of these.

In embodiments, each first particle has between about 2 and about 1,000 nucleophilic moieties or electrophilic moieties, e.g., between about 10 and about 500 or between about 25 and about 250.

In embodiments, the composite further includes second, third, fourth or even fifth particles different than the first particles. In some instances, the other particles are not covalently bonded to the first polymeric material. For example, the other particles can be particles of a metal, a metal oxide (e.g., titanium dioxide), a metalloid oxide (e.g., silicon dioxide), a clay (e.g., kaolin), a ceramic (e.g., silicon carbide or titanium nitride) or a crosslinked polymeric material different from the first polymeric material. In a particular embodiment the other particles are each in the form of an allotrope of carbon extending from a substrate, which is or includes a clay material. Such particles can be advantageous because the clay-containing particles can be easier to disperse and can have a reduced tendency to aggregate. Such particles can also provide mechanical interlocking within the matrix, providing enhanced mechanical properties to the composite. In addition, the clay can improve the biocompatibility of the composite and can increase its ion-exchange capacity.

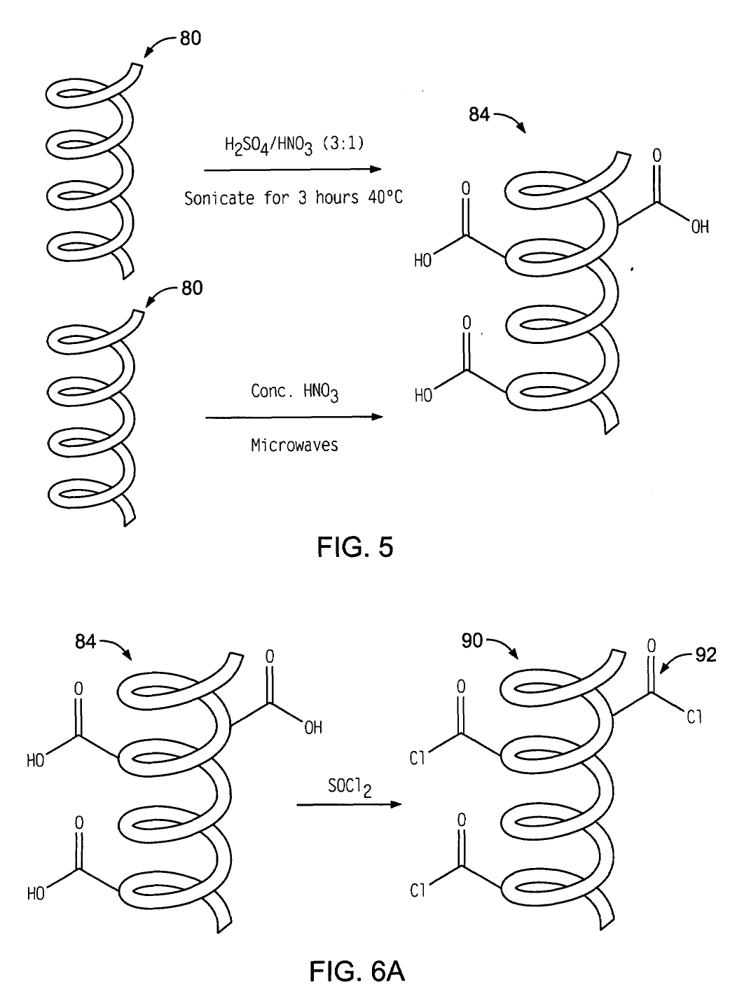

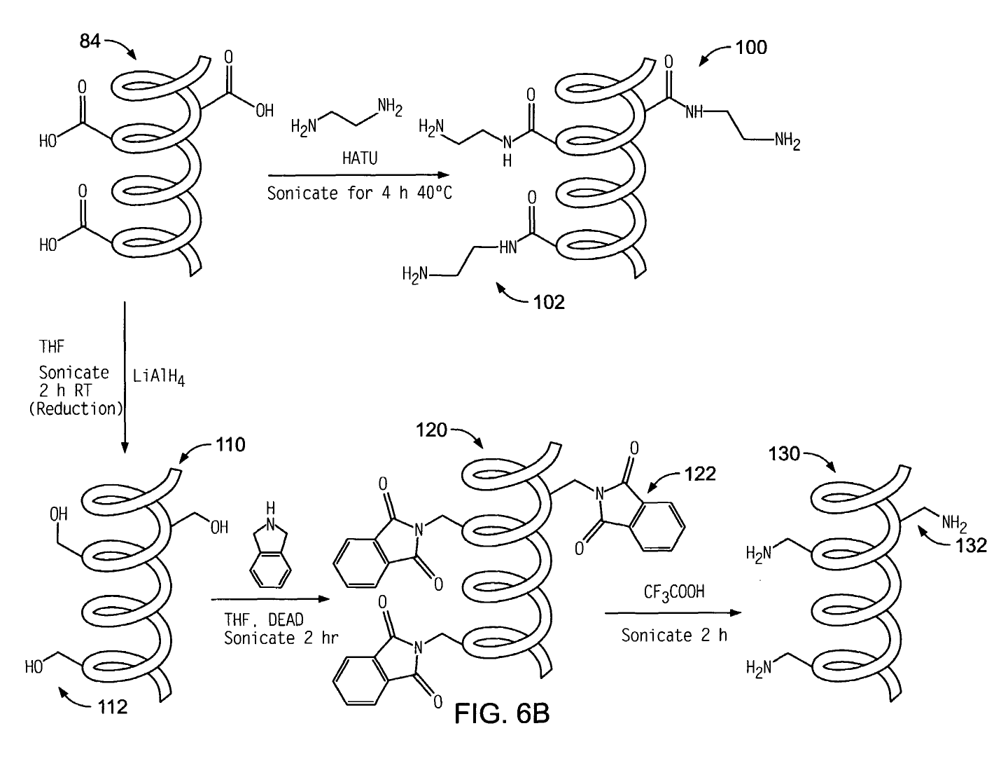

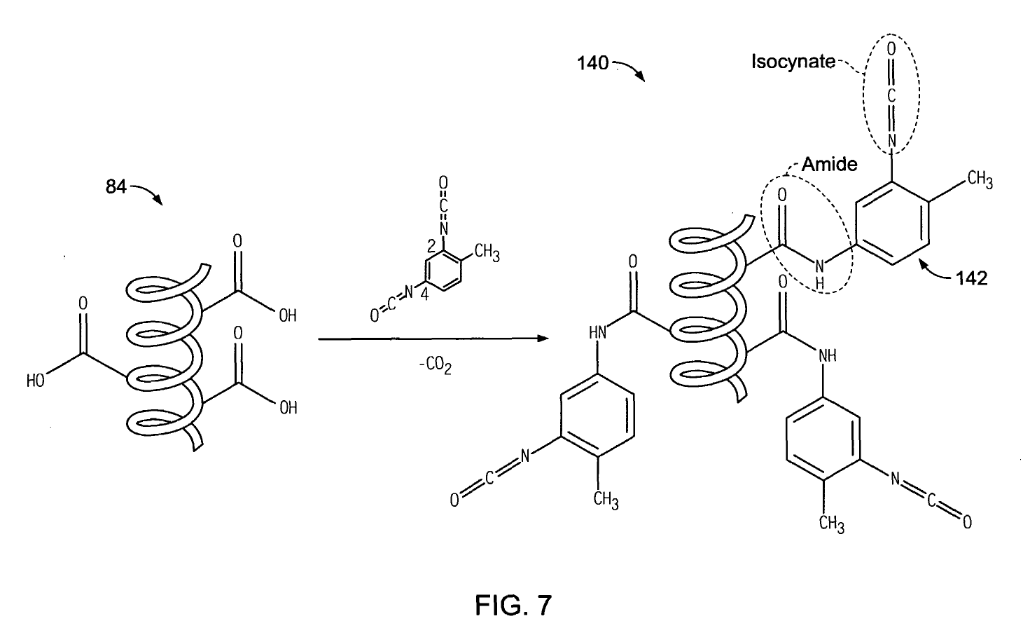

FIGS. 5-7 illustrate techniques for functionalizing allotropes of carbon, such as carbon coils. FIG. 5, in particular, shows that a carbon coil 80 can be converted into a carbon coil 84 functionalized with carboxylic acid groups by (A) reacting carbon coils 80 with a 3:1 mixture of sulfuric acid/nitric acid with sonication for 3 hours at 40° C.; or (B) by reacting carbon coils 80 with concentrated nitric acid while irradiating with microwaves. FIG. 6A shows that the carbon coils 84 can be converted to carbon coils 90 functionalized with acid chloride groups 92 by treatment of carbon coils 84 with thionyl chloride (SOCl₂). FIG. 6B shows that carbon coils 84 can be converted to carbon coils 100 functionalized with primary amino-amide groups 102 by reacting carbon coils 84 with ethylene diamine and N-[(dimethylamino)-1H-1,2,3-triazolo[4,5,6]pyridin-1-ylmethylene]-N-methylmethanaminium hexafluorophosphate N-oxide (HATU) with sonication for 4 hours at 40° C. FIG. 6B also shows that carbon coils 84 can be reduced in the presence of lithium aluminum hydride in THF with sonication for 2 hours at room temperature to the corresponding carbon coil 110 functionalized with primary alcohol moieties 112. In addition, FIG. 6A shows the carbon coils 110 can be converted carbon coils 120 functionalized with cyclic amide moieties 122 by treatment with phthalimide and diethylazodicarboxylate (DEAD) in THF with sonication, and that carbon coils 120 can be hydrolyzed with trifluoroacetic acid under sonication for 2 hours to carbon coils 130 functionalized with primary amino groups 132. FIG. 7 shows, in particular, that carbon coil 84 functionalized with carboxylic acid groups can react as a nucleophile when it reacts with toluene-2,4-di-isocyanate to produce a carbon coil 140 with amide-isocyanate functionalization 142. All of the functionalized carbon coils described above can be used as the basis for incorporation of carbon coils into a polymeric matrix, as will be further described below using some specific examples.

Various techniques for functionalizing allotropes of carbon are discussed in Wang et al, Carbon 43 (2005), 1015-1-20; Ramanathan et al., Chem. Mater. (2005), 17, 1290-1295; Zhao et al., Journal of Solid State Chemistry (2004), 177, 4394-4398; and in Jung et al., Materials Science and Engineering (2004), C24, 117-121.

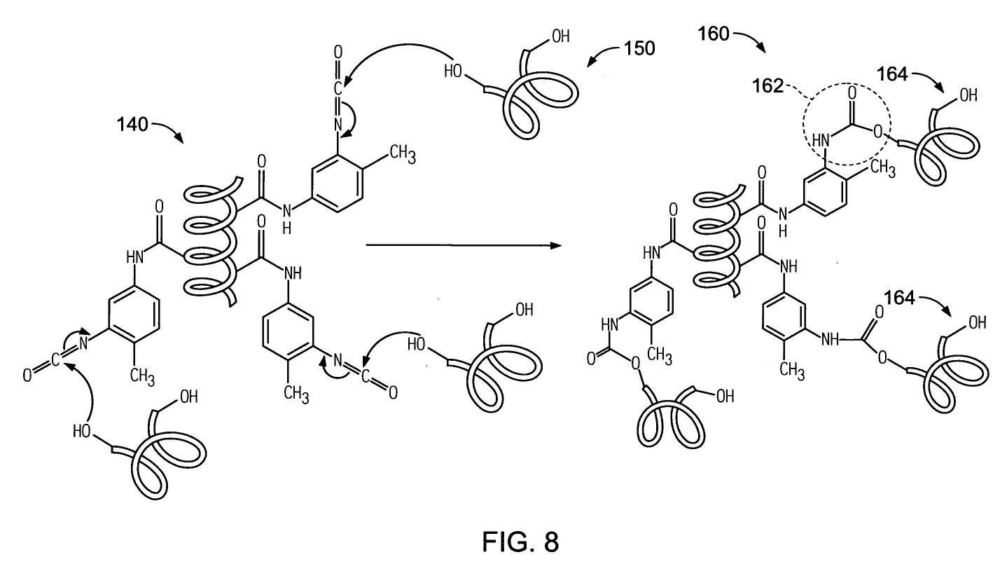

Referring now to FIG. 8, carbon coil 140 with amide-isocyanate functionalization can react with a polyol 150, e.g., having 2 or more hydroxyl groups, e.g., 3-10 hydroxyl groups, to produce pre-polymer 160 having urethane linkages 162 and terminal hydroxyl groups 164.

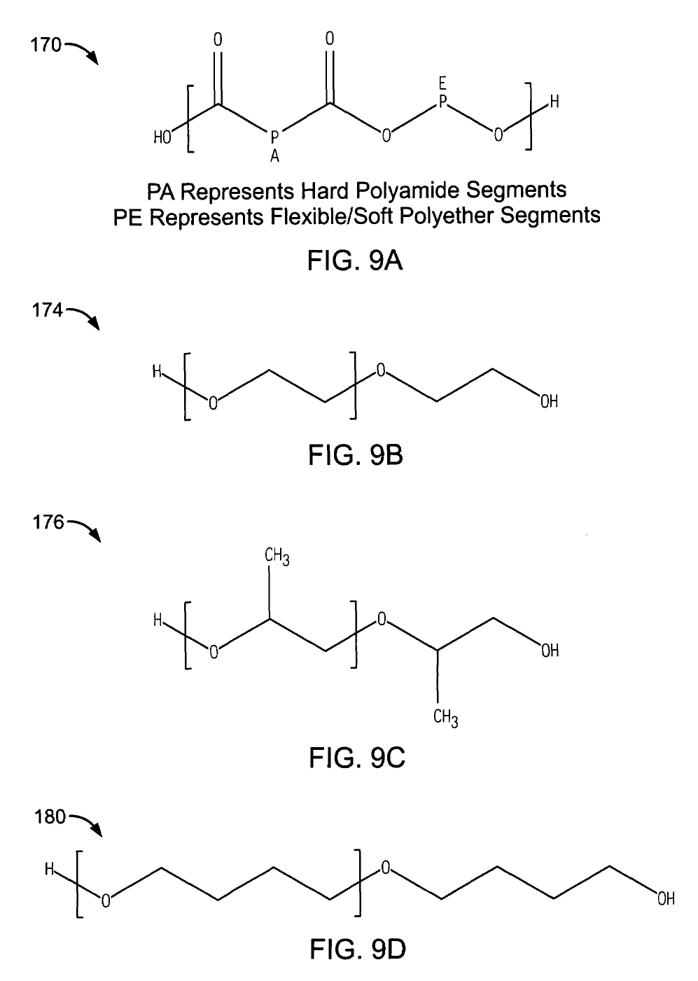

FIGS. 9A-9D show various polyols. In particular, FIGS. 9A-9D show representations of a polyetheramide (170, FIG. 9A) having hard polyamide (PA) segments and soft/flexible polyether (PE) segments; a di-hydroxyl terminated PEG (174, FIG. 9B); a di-hydroxyl terminated polypropylene glycol (176, FIG. 9C); and a di-hydroxyl terminated polytetramethylene glycol (180, FIG. 9D).

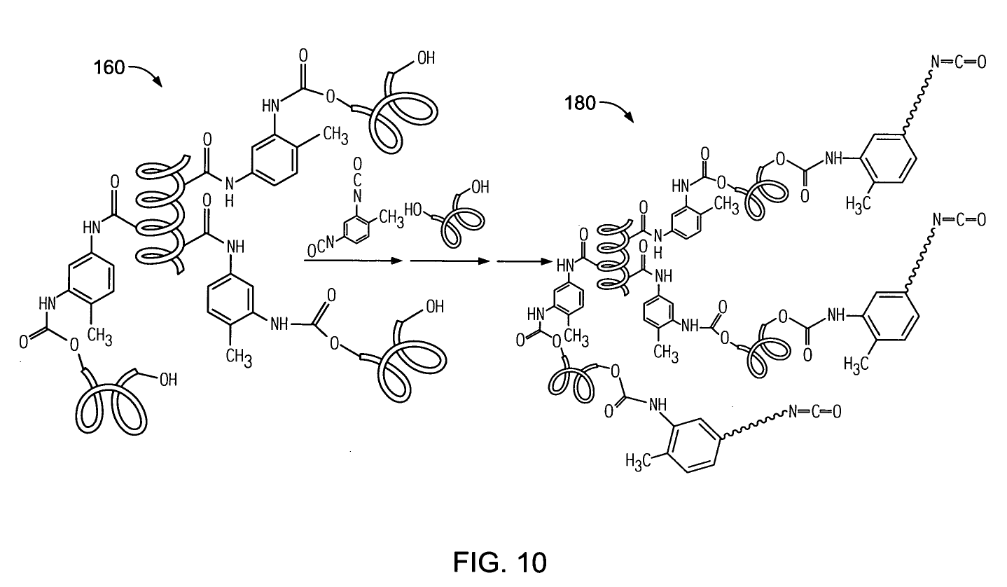

Referring now to FIG. 10, pre-polymer 160 can be further reacted with monomeric isocyanate, such as toluene-2,4-di-isocyanate, and one or more polyols to produce a higher molecular weight polymer 180 that is terminated with reactive isocyanate groups. Since high polymer 180 is isocyanate terminated, it is reactive (often called “living”) and can be reacted with other monomers and polymers, e.g., that include nucleophilic portions.

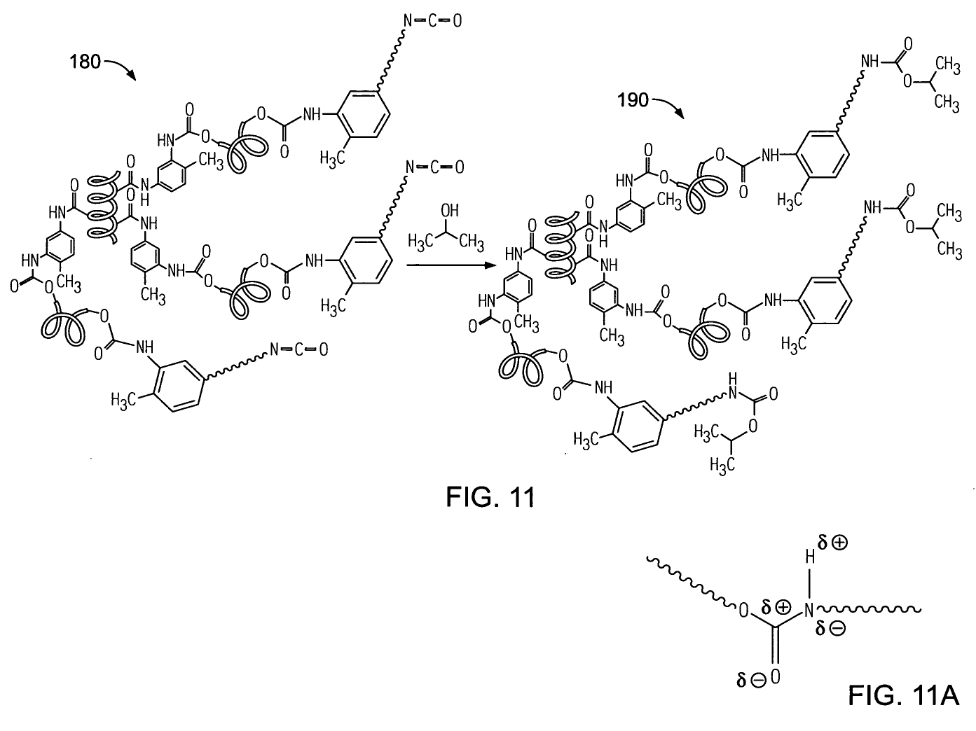

Reactive polymer 180 can be made less so by quenching the terminal isocyanate groups with a cap. In particular, FIG. 11 shows that polymer 180 can be converted into a lower reactivity polymer 190 by reaction with isopropanol. Methods of quenching reactive isocyanate end groups with isopropanol are discussed in Yilgor et al., Polymer (2004), 45, 5829-5836.

It should be noted, and by reference to FIG. 11A, high polymer 190 includes urethane and amide linkages that can act as hydrogen-bonding acceptor/donor sites. Because of this functionality, a polymer such as 190 can interact with itself or other polymers having hydrogen-bonding acceptor/donor portions. For example, a polymer such as 190 can be reversibly “crosslinked” by hydrogen bonding with itself or with one or more other polymers. This can enhance the properties of the resulting composites, tensile strength and flexural modulus.

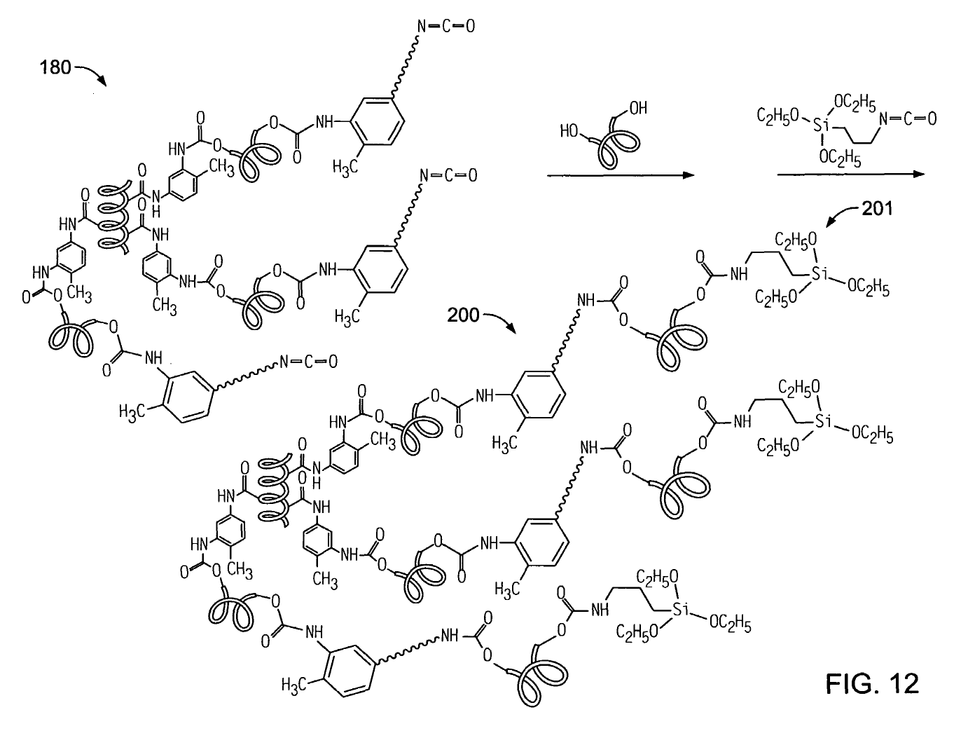

Referring to FIG. 12, reactive polymer 180 can be reacted with additional polyol, followed by reaction with an isocyanate-terminated trialkoxysilane, such as 3-isocyanatopropyl-triethoxysilane, to produce a high polymer 200 having trialkoxysilane-terminal groups 201. Such polymers can be crosslinked through the terminal trialkoxysilane groups, e.g., by treatment with water. Crosslinking of polymers having terminal trialkoxysilane groups is discussed in Honma et al., Journal of Membrane Science (2001), 185, 83-94.

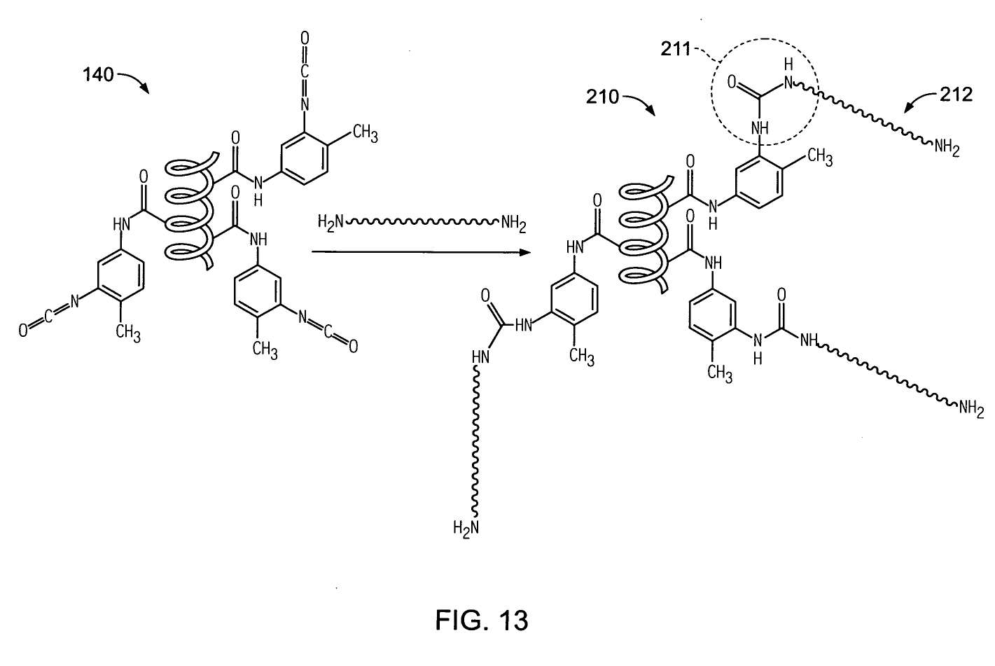

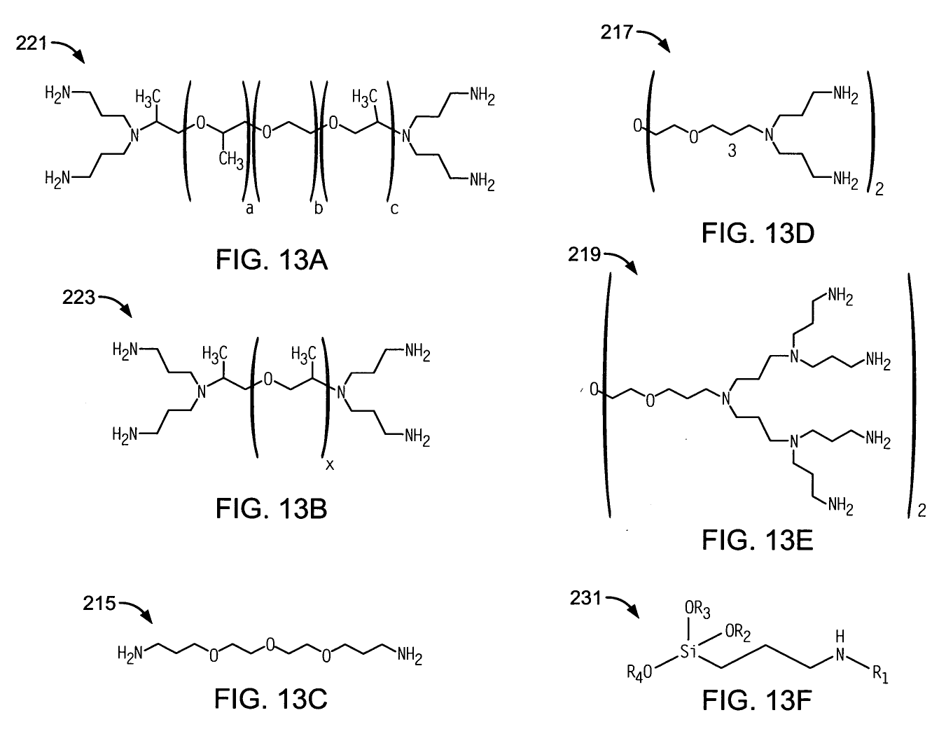

Referring now to FIG. 13, carbon coils 140 with amide-isocyanate functionalization can react with polyamines to produce a high molecular weight polymer 210 having urea linkages 211 and terminal amino groups 212. The polyamine can have 2 or more amino groups, e.g., 3-10 amino groups. For example, the polyamine can be a polymer, e.g., an α,ω-diamino polyether such as 221 (FIG. 13A) or 223 (FIG. 13B), or a monomer that is terminated with primary amino groups, such as 215 (FIG. 13C), 217 (FIG. 13D) or 219 (FIG. 13E). Various polyamines are discussed in Tetrahedron Letters (2005), 46, 2653-2657.

Polymer 210 can react with additional monomeric isocyanate, followed by reaction with an amino-terminated trialkoxysilane, such as 231 (FIG. 13F), to produce a high polymer having trialkoxysilane-terminal groups. In embodiments, R₁ of 231 is, e.g., H, methyl, ethyl, n-propyl or isopropyl, and R₂-R₄ of 231 are each methyl, ethyl, n-propyl or isopropyl. Such polymers can be crosslinked through the terminal trialkoxysilane groups, e.g., by treatment with water.

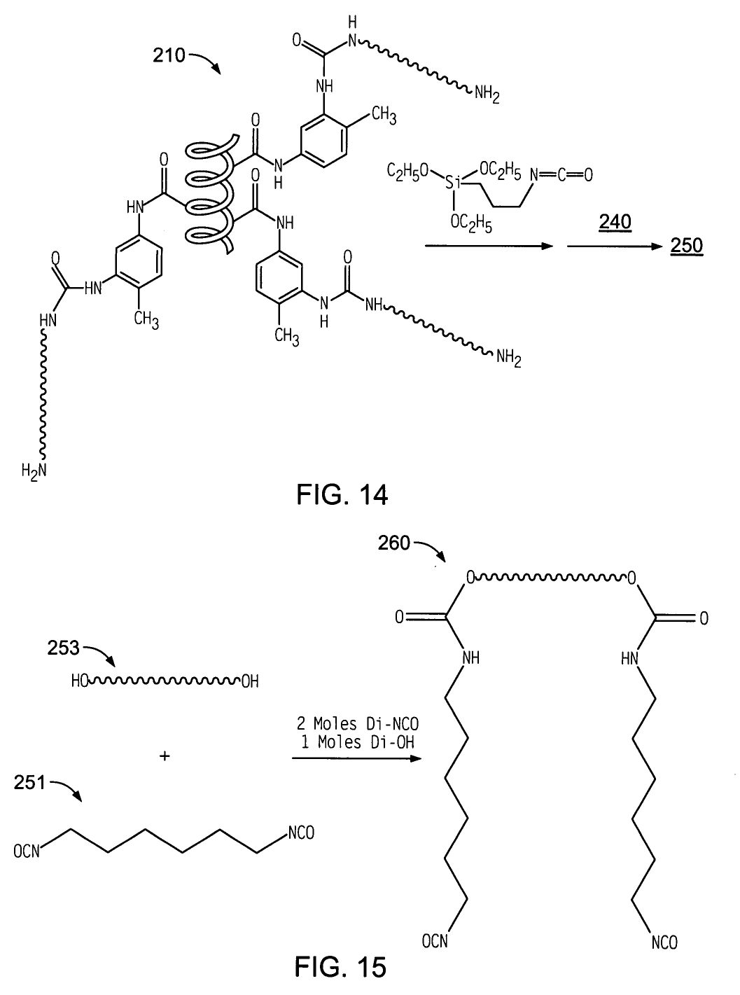

Referring to FIG. 14, primary amino group-functionalized polymer 210 can be reacted with an isocyanate-terminated tri-alkoxysilane, such as 3-isocyanato-propyltriethoxysilane, to produce a high polymer 240 having trialkoxysilane-terminated groups, which can be crosslinked through the terminal trialkoxysilane groups, e.g., by treatment with water, to produce a crosslinked hybrid polymer 250.

Referring now to FIG. 15, in a particular embodiment, 1 mole of a α,ω-polyol 253 is reacted with two moles of hexamethylene di-isocyanate to produce a reactive pre-polymer 260 having terminal isocyanate groups. Such a pre-polymer can be reacted with other polymers, such as polyols or polyamines, to produce high polymers.

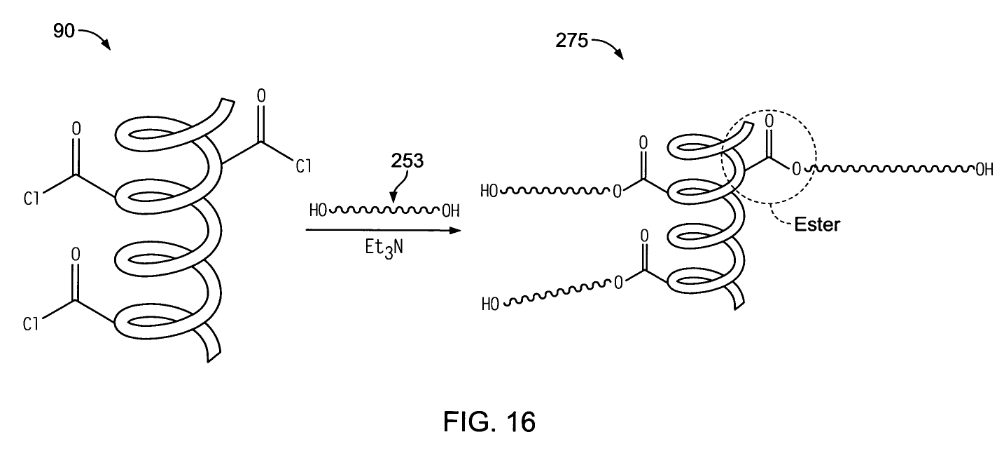

Referring now to FIG. 16, acid halide-functionalized coils 90 can be reacted with a polyol 253 to produce a polymer 275 that includes an ester linkage.

The composite materials formed by any of the methods described herein can have a high tensile strength, e.g., the tensile strength can be greater than about 40 MPa, e.g., greater than about 50 MPa, 75 MPa, 100 MPa, or even greater than about 150 MPa. In addition, the composite material can have a high electrical conductivity, e.g., greater than about 50 S/cm, e.g., greater than about 60 S/cm, 75 S/cm, 100 S/cm, 150 S/cm, 200 S/cm, even greater than about 300 S/cm.

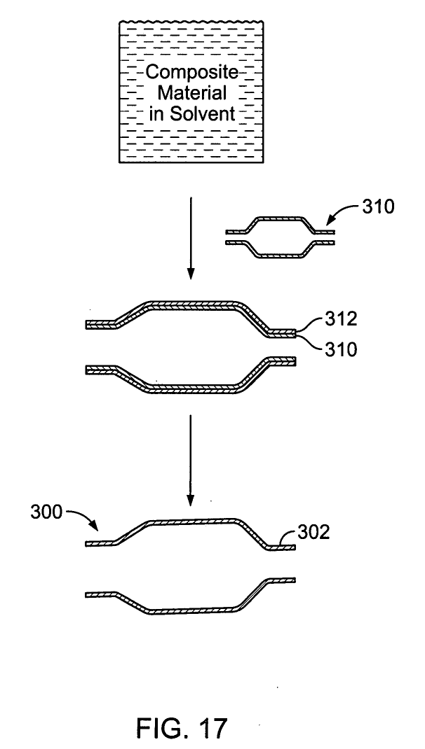

Referring now to FIG. 17, a balloon 300 that includes a wall 302 formed of a composite material can be made by depositing a solution containing the composite material onto a substrate 310, e.g., by spraying the solution onto the substrate. Substrate 310 can be, e.g., made of ice. Once the composite has been deposited, the solvent can removed from the deposited solution, forming a layer 312 about the substrate 310. After the solvent is removed and the composite is set, the substrate can be removed. In instances in which the substrate is ice, the substrate can be removed by melting or freeze-drying. After removal of the substrate, balloon 300 is provided. If desired, the balloon can be coated with more composite material, or another material, forming multiple layers of the same or different materials.

Porous balloons and/or catheters can be made by treating a composite that includes unreacted isocyanate groups with water. If desired, the composite can have interconnected voids. For example, the voids can have a maximum dimension that is greater than 500 nm, e.g., greater than 750 nm, 1,000 nm, 1,500 nm, or even greater than 2,500 nm. The voids can provide a porosity that is, e.g., greater than 75 percent, e.g., greater than 80 percent, 85 percent, 90 percent, or even greater than 95 percent, as measured using mercury porosimetry.

In any of the above embodiments, the wall can include a therapeutic agent therein and/or thereon. In some desirable implementations, the wall is porous and filled with a therapeutic agent so that the agent can be delivered from the balloon during deployment of a medical device.

Electroporation and iontophoresis can be used to assist in the delivery of a therapeutic agent. For example, when a therapeutic agent is utilized in a conductive composite, delivery of the therapeutic can be aided by applying an electric field to the conductive composite, e.g., between about 5 V/cm and about 2.5 kV/cm, between about 25 V/cm and about 1.5 kV/cm or between about 50 kV/cm and about 1 kV/cm. In some embodiments, the electric field is applied in a pulsing manner. For example, the pulse length can be from about 50 μs to about 30 ms, from about 100 μs to about 25 ms or from about 150 μs to about 20 ms. Generally, electroporation is described by Davalos et al., Microscale Thermophysical Engineering, 4:147-159 (2000). A power supply for a pulsed power supply for electroporation has been described by Grenier, a thesis presented to the University of Waterloo, Ontario, Canada, in a work entitled “Design of a MOSFET-Based Pulsed Power Supply for Electroporation” (2006).

In certain implementations, charged biofunctional moieties are disposed in a porous outer layer of a balloon and an electric field is utilized to drive the moieties out of the balloon. In other certain implementations, a double-layered balloon is utilized, the outer layer being a ion exchange membrane and the inner being any of the materials described herein. Between the two layers is an electrolyte solution containing the charged therapeutic moieties, e.g., molecules. An electric field can be utilized to pass the therapeutic moieties into surrounding tissues.

In general, the therapeutic agent can be a genetic therapeutic agent, a non-genetic therapeutic agent, or cells. Therapeutic agents can be used singularly, or in combination. Therapeutic agents can be, e.g., nonionic, or they may be anionic and/or cationic in nature. A preferred therapeutic agent for some embodiments is one that inhibits restenosis. A specific example of one such therapeutic agent that inhibits restenosis is paclitaxel or derivatives thereof,

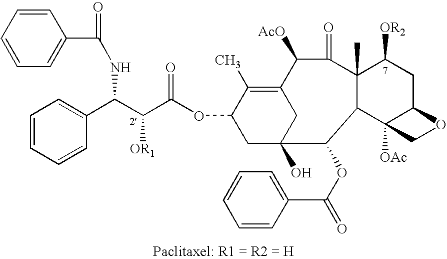

e.g., docetaxel. Soluble paclitaxel derivatives can be made by tethering solubilizing moieties off the 2′ hydroxyl group of paclitaxel, such as —COCH₂CH₂CONHCH₂CH₂(OCH₂)ₙOCH₃ (n being, e.g., 1 to about 100 or more). Li et al., U.S. Pat. No. 6,730,699 describes additional water soluble derivatives of paclitaxel.

Exemplary non-genetic therapeutic agents include: (a) anti-thrombotic agents such as heparin, heparin derivatives, urokinase, PPack (dextrophenylalanine proline arginine chloromethylketone), and tyrosine; (b) anti-inflammatory agents, including non-steroidal anti-inflammatory agents (NSAID), such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, rapamycin (sirolimus), biolimus, tacrolimus, everolimus, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines, (r) hormones; and (s) antispasmodic agents, such as alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, tropenzile, trospium chloride, xenytropium bromide, ketorolac, and pharmaceutically acceptable salts thereof.

Exemplary genetic therapeutic agents include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or micro particles, with and without targeting sequences such as the protein transduction domain (PTD).

### Other Embodiments

A number of embodiments of have been described. Nevertheless, it will be understood that various modifications may be made without departing from the spirit and scope of the disclosure.

Balloons and/or catheters can have walls that include more than 1 layer. For example, a wall can have 2, 3, 4, 5, 7, 9, 11, 13, 15 or more layers, e.g., 21 layers.

While embodiments have been illustrated in which an entire wall, such as a wall of a balloon, is formed of the composite, in some embodiments, only a portion of the wall is made of the composite. Still other embodiments are in the following claims.

What is claimed is:

1. A medical balloon or catheter comprising a wall comprising a composite material comprising a first polymeric material and first particles comprising an allotrope of carbon, wherein at least some of the first particles are covalently bonded to the first polymeric material.

2. The balloon or catheter of claim 1, wherein the first polymeric material comprises segments selected from the group consisting of polyethers, polyurethanes, polyether-polyurethane copolymers, polyamides, polyether-polyamide copolymers, polyureas, polyether-polyurea copolymers, polyamines, polyesters, polysiloxanes, and mixtures thereof.

3. The balloon or catheter of claim 1, wherein the first polymeric material is a thermoplastic material.

4. The balloon or catheter of claim 1, wherein the composite material further comprises a second polymeric material different from the first polymeric material.

5. The balloon or catheter of claim 1, wherein the first particles are in the form of coils.

6. The balloon or catheter of claim 1, wherein the allotrope of carbon is selected from the group consisting of graphite, C60, C70, a single wall carbon tube, multi-wall carbon tube, amorphous carbon, a carbon coil, a carbon helix, carbon rope, carbon fiber, and mixtures thereof.

7. The balloon or catheter of claim 1, wherein each first particle is has a length-to-diameter ratio of greater than 5.

8. The balloon or catheter of claim 1, wherein each first particle has a maximum dimension not exceeding 1,000 nm.

9. The balloon or catheter of claim 1, wherein the first particles are discrete and spaced apart throughout the composite.

10. The balloon or catheter of claim 1, wherein the composite further comprises second particles different than the first particles, the second particles not being covalently bonded to the first polymeric material.

11. The balloon or catheter of claim 10, wherein the second particles are selected from the group consisting of metals, metal oxides, metalloid oxides, clays, ceramics, and mixtures thereof.

12. The balloon or catheter of claim 1, wherein at least about 2.5 percent of the total number of the first particles are covalently bonded to the first polymeric material.

13. The balloon or catheter of claim 1, wherein the composite comprises from about 5 percent by weight to about 60 percent by weight of the first particles comprising the allotrope of carbon.

14. The balloon or catheter of claim 1, wherein the first particles are covalently bonded to the first polymeric material by a covalent bond connecting a carbon atom of the allotrope of carbon and the first polymeric material.

15. The balloon or catheter of claim 14, wherein the first particles are covalently bonded to the first polymeric material by a reaction between a nucleophilic moiety covalently attached to the allotrope of carbon and a complementary electrophilic moiety covalently attached to the first polymeric material or a pre-first polymeric material.

16. The balloon or catheter of claim 15, wherein the nucleophilic moiety comprises a nucleophile selected from the group consisting of an amino group, a hydroxyl group, a thiol group, conjugate bases thereof, and mixtures thereof.

17. The balloon or catheter of claim 15, wherein each first particle has between about 2 and about 1,000 nucleophilic moieties.

18. The balloon or catheter of claim 1, wherein at least some of the first particles comprising the allotrope of carbon further comprise a substrate bonded to the allotrope of carbon.

19. The balloon or catheter of claim 18, wherein the support comprises a clay comprising an allotrope of carbon-forming catalyst thereon and/or therein.

20. The balloon or catheter of claim 19, wherein the clay is selected from the group consisting of kaolinite, montmorillonite-smectite, illite, chlorite, and mixtures thereof.

21. The balloon or catheter of claim 1, wherein the wall comprises multiple layers.

22. The balloon or catheter of claim 21, wherein the composite material is in each layer of said multiple layers, and wherein each layer is integral with its neighbor.

23. The balloon or catheter of claim 21, wherein the composite material is in a single layer, the single layer being integral with a second layer formed of a material different than said composite material.

24. The balloon or catheter of claim 1, wherein the wall includes a therapeutic agent therein and/or thereon.

25. A method of making a medical balloon or catheter, the method comprising:
forming a wall comprising a composite material comprising a first polymeric material and first particles comprising an allotrope of carbon, wherein at least some of the first particles are covalently bonded to the first polymeric material.

26. The method of claim 25, wherein the wall is formed by providing a substrate; and depositing the composite material onto the substrate.

27. The method of claim 26, further comprising removing the substrate.

28. The method of claim 26, wherein the composite material is deposited by spraying a solution of the composite material onto the substrate.

29. A medical balloon or catheter comprising a wall comprising a composite material comprising a polymeric material and particles, wherein the particles comprise a substrate comprising a material comprising a clay, and an allotrope of carbon extending from the substrate.

30. The balloon or catheter of claim 29, wherein the clay has an allotrope of carbon-forming catalyst thereon and/or therein.

31. The balloon or catheter of claim 29, wherein the clay is selected from the group consisting of kaolinite, montmorillonite-smectite, illite, chlorite, and mixtures thereof.

32. The balloon or catheter of claim 30, wherein the allotrope of carbon-forming catalyst comprises group 8 or group 9 element.
